(19) United States
(12) Patent Application Publication
(10) Pub. No.: US 2026/0015605 A1
(43) Pub. Date: Jan. 15, 2026

(54) HIGH-PURITY PDRN EXTRACT DERIVED FROM MICROALGAE AND MANUFACTURING METHOD THEREOF

(71) Applicant: AMOREPACIFIC CORPORATION, Seoul (KR)

(72) Inventors: Hyuk KIM, Yongin-si (KR); Jaeyoung KO, Yongin-si (KR); Gibaeg NAM, Yongin-si (KR); Kilsun MYOUNG, Yongin-si (KR); Heungsoo BAEK, Yongin-si (KR)

(73) Assignee: AMOREPACIFIC CORPORATION, Seoul (KR)

(21) Appl. No.: 19/234,658

(22) Filed: Jun. 11, 2025

(30) Foreign Application Priority Data
Jul. 15, 2024 (KR) 10-2024-0093298

(51) Int. Cl. C12N 15/10; A61K 8/60; A61Q 19/00; C12N 1/12; C12R 1/89

(52) U.S. Cl. CPC C12N 15/1017; A61K 8/606; A61Q 19/00; C12N 1/12; A61K 2800/805; C12R 2001/89

(57) ABSTRACT

The present disclosure relates to a high-purity PDRN extract derived from microalgae and a preparation method thereof. Specifically, the high-purity PDRN extract derived from microalgae according to the present disclosure is easy to produce and highly sustainable by using microalgae capable of being mass-cultivated at any time without external environmental or time constraints as a raw material, is safe due to no risk of human infection by exogenous viruses, and can also be excellent in skin absorption when applied to products such as cosmetics due to its low molecular weight.

### DESCRIPTION OF GOVERNMENT-SPONSORED RESEARCH

This research was conducted under the supervision of Amorepacific Corporation and with the support of the Ministry of Oceans and Fisheries' Marine Bio Raw Material Formulation Technology Development Project (development of mass production process for microalgae-derived PDRN and standardization of highly effective skin cosmetic materials, Project Unique Number: 1525014981).

### CROSS REFERENCE TO RELATED APPLICATION

The present application claims priority to Korean Patent Application No. 10-2024-0093298, filed Jul. 15, 2024, the entire contents of which are hereby incorporated by this reference.

### BACKGROUND OF THE INVENTION

### Field of the Invention

The present disclosure relates to a high-purity PDRN extract derived from microalgae and a preparation method thereof.

### Description of the Related Art

Polydeoxyribonucleotide (PDRN) is a tissue regeneration active material, and a polymer with a molecular weight ranging from 50 to 1,500 kDa made from DNA that promotes the self-regeneration of damaged cells and tissues. Unlike common DNA, PDRN exhibits pharmacological activity without transmitting genetic information, and stimulates the A2 receptor, which is a signal transmitter for skin regeneration, promoting the secretion of various growth factors, generating capillaries through vascular endothelial growth factors (VEGFs), improving blood circulation, exerting anti-inflammatory actions, and preventing capillary leakage.

Further, PDRN is constantly present in human cells and physiologically stimulates the regeneration and metabolic activity of fibroblasts to help maintain the best condition. PDRN has received much attention as a result of research showing that it is effective in treating wounds caused by burns and the like by promoting skin regeneration without any particular side effects, and it is known to have anti-inflammatory effects and the like by shortening wound healing time and activating cell regeneration through rapid tissue regeneration, activating cell regeneration, simultaneously promoting the production of collagenous and non-collagenous proteins, and promoting the differentiation of various cells (stem cells, fibroblasts, osteoblasts, chondrocytes, and the like).

Since such PDRNs are DNA polymers and biological components, there is an advantage in that there are no allergic reactions or rejection reactions in the body that occur in synthetic substances, and the like. Most PDRN raw materials are derived from fish, and even though DNA is extracted from the semen and testes of fish such as salmon and then PDRN is mass-purified therefrom, there is a possibility of mass fish deaths and infection, and there are also time constraints such as extraction limited to seasonal cycles, so that there is a need for a method of purifying plant-based PDRN that can be mass-grown and harvested at any time as an alternative. In particular, the extraction of PDRNs from non-animal plants would avoid the possibility of contamination with exogenous viruses that may be zoonotic or human-transmissible due to the incorporation of viruses derived from animal tissue.

### SUMMARY OF THE INVENTION

It is an object of the present disclosure to provide a high-purity PDRN extract, which is easy to produce and highly sustainable by using microalgae capable of being mass-cultivated at any time without external environmental or time constraints as a raw material, and which is safe because it does not pose a risk of human infection by exogenous viruses, and a preparation method thereof.

To achieve the aforementioned object, an embodiment of the present invention provides a high-purity polydeoxyribonucleotide (PDRN) extract derived from microalgae, wherein a DNA content relative to the total dry weight of the PDRN extract is 49 wt % or more.

In addition, another embodiment of the present invention provides a cosmetic composition including the PDRN extract as an active ingredient.

Furthermore, still another embodiment of the present invention provides a method for preparing a high-purity PDRN extract derived from microalgae, the method including: (1) adding an anionic surfactant and a metal salt to a microalgae solution and stirring the resulting mixture to obtain a cytolysate; (2) adding a metal salt to a filtrate obtained by centrifuging and filtering the cytolysate of step (1) to stir the resulting mixture; (3) a first purification step of adding a C1-C4 alcohol to a filtrate obtained by centrifuging and filtering the stirred solution of step (2) and centrifuging the resulting mixture to obtain a precipitate; (4) a second purification step of adding a C1-C4 alcohol to the precipitate of step (3) and centrifuging the resulting mixture to obtain a precipitate; and (5) freeze-drying the precipitate of step (4).

The high-purity PDRN extract derived from microalgae according to the present disclosure is easy to produce and highly sustainable by using microalgae capable of being mass-cultivated at any time without external environmental or time constraints as a raw material, is safe due to no risk of human infection by exogenous viruses, and can also be excellent in skin absorption when applied to products such as cosmetics due to its low molecular weight.

### BRIEF DESCRIPTION OF THE DRAWINGS

FIG. 1 illustrates the PDRN extraction process according to Preparation Example 1.

FIG. 2 illustrates the results of confirming the cell lysis effect depending on the concentration of a surfactant according to Test Example 1.

FIG. 3 illustrates the PDRN extraction process and the cell lysis effect according to Preparation Example 2.

FIG. 4 illustrates the PDRN extraction process according to Preparation Example 3.

FIG. 5 illustrates the PDRN extraction process according to Preparation Example 4.

FIGS. 6A and 6B illustrate the results of confirming the size and purity of DNA of PDRN derived from golden chlorella (Chlorella protothecoides), green chlorella (Chlorella vulgaris) and white chlorella (Chlorella protothecoides), respectively.

FIG. 7 illustrates the results of confirming the DNA size of high-purity PDRN derived from golden chlorella.

### DETAILED DESCRIPTION OF THE INVENTION

Hereinafter, the present invention will be described in detail.

In one aspect, the present invention may relate to a high-purity polydeoxyribonucleotide (PDRN) extract derived from microalgae, wherein a DNA content relative to the total dry weight of the PDRN extract is 49 wt % or more.

In an embodiment, the PDRN extract may have a DNA content of 49 wt % or more, 50 wt % or more, 51 wt % or more, 52 wt % or more, 53 wt % or more, 54 wt % or more, 55 wt % or more, 56 wt % or more, 57 wt % or more, 58 wt % or more, 59 wt % or more, 60 wt % or more, 61 wt % or more, 62 wt % or more, 63 wt % or more, 64 wt % or more, 65 wt % or more, 66 wt % or more, 67 wt % or more, 68 wt % or more, 69 wt % or more, 70 wt % or more, 71 wt % or more, 72 wt % or more, 73 wt % or more, 74 wt % or more, 75 wt % or more, 76 wt % or more, 77 wt % or more, 78 wt % or more, 79 wt % or more, 80 wt % or more, 81 wt % or more, 82 wt % or more, 83 wt % or more, 84 wt % or more, 85 wt % or more, 86 wt % or more, 87 wt % or more, 88 wt % or more, 89 wt % or more, 90 wt % or more, 91 wt % or more, 92 wt % or more, 93 wt % or more, 94 wt % or more, 95 wt % or more, 96 wt % or more, 97 wt % or more, 98 wt % or more, or 99 wt % or more relative to the total dry weight of the PDRN extract.

In an embodiment, at least 90% of the DNA in the PDRN extract may be low molecular weight DNA having a molecular weight of 20 kDa or less.

Specifically, at least 90%, 91%, 92%, 93%, 94%, 95%, 96%, 97%, 98% or 99% of the DNA in the PDRN extract may have a molecular weight of 20 kDa or less, 19 kDa or less, 18 kDa or less, 17 kDa or less, 16 kDa or less, 15 kDa or less, 14 kDa or less, 13 kDa or less, or 12 kDa or less, and further, at least 90%, 91%, 92%, 93%, 94%, 95%, 96%, 97%, 98% or 99% of the DNA in the PDRN extract may have a molecular weight of 10 kDa or more, 11 kDa or more, 12 kDa or more, 13 kDa or more, 14 kDa or more, 15 kDa or more, 16 kDa or more, 17 kDa or more, 18 kDa or more, or 19 kDa or more.

In an embodiment, the PDRN extract may be in the form of a white powder.

In an embodiment, the microalgae may be one or more selected from among Chlorella, Spirulina, Dunaliella, Haematococcus, Euglena, Nannochloropsis, Nostoc, and Schizochytrium.

The chlorella may be one or more species selected from among, for example, golden chlorella (Chlorella protothecoides), green chlorella (Chlorella vulgaris), and white chlorella (Chlorella protothecoides), and may be particularly golden chlorella (Chlorella protothecoides).

In another aspect, the present invention may relate to a method for preparing a high-purity PDRN extract derived from microalgae.

In an embodiment, the method may include: (1) adding an anionic surfactant and a metal salt to a microalgae solution and stirring the resulting mixture to obtain a cytolysate; (2) adding a metal salt to a filtrate obtained by centrifuging and filtering the cytolysate of step (1) to stir the resulting mixture; (3) a first purification step of adding a C1-C4 alcohol to a filtrate obtained by centrifuging and filtering the stirred solution of step (2) and centrifuging the resulting mixture to obtain a precipitate; and (4) a second purification step of adding a C1-C4 alcohol to the precipitate of step (3) and centrifuging the resulting mixture to obtain a precipitate.

In an embodiment, the microalgae solution of step (1) may be a mixture of a microalgae powder and purified water, and the weight of the microalgae solution may be 20-fold or more than the weight of the microalgae powder.

In an embodiment, the microalgae of step (1) may be one or more selected from among golden chlorella (Chlorella protothecoides), green chlorella (Chlorella vulgaris), white chlorella (Chlorella protothecoides), Spirulina, Dunaliella, Haematococcus, Euglena, Nannochloropsis, Nostoc, and Schizochytrium.

In an embodiment, the anionic surfactant in step (1) may be sodium dodecyl sulfate (SDS).

In an embodiment, the method may further include repeatedly applying an ultra-high pressure of 500 to 2,000 bar to the mixture of step (1) three or more times to obtain cell debris, prior to step (2).

Specifically, the pressure applied to the mixture of step (1) may be 500 bar or more, 600 bar or more, 700 bar or more, 800 bar or more, 900 bar or more, 1,000 bar or more, 1,100 bar or more, 1,200 bar or more, 1,300 bar or more, 1,400 bar or more, 1,500 bar or more, 1,600 bar or more, 1,700 bar or more, or 1,800 bar or more, or 1,900 bar or more, and in addition, the pressure applied to the mixture of step (1) may be 2,000 bar or less, 1,900 bar or less, 1,800 bar or less, 1,700 bar or less, 1,600 bar or less, 1,500 bar or less, 1,400 bar or less, 1,300 bar or less, 1,200 bar or less, 1,100 bar or less, 1,000 bar or less, 900 bar or less, 800 bar or less, 700 bar or less, or 600 bar or less.

In an embodiment, the metal salt may be sodium chloride (NaCl).

In an embodiment, the concentration of the metal salt added in step (2) may be 2-fold or more than the concentration of the metal salt added in step (1).

In an embodiment, the ratio of the filtrate to the C1-C4 alcohol added to the filtrate in step (3) may be 1:1 to 3 by volume. Specifically, the volume ratio of the filtrate: the C1-C4 alcohol added to the filtrate may be 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, or 1:3.

In an embodiment, the metal salt and the cationic surfactant may be added together in step (2).

In an embodiment, the cationic surfactant may be cetyl trimethyl ammonium bromide (CTAB).

In an embodiment, step (4) may include adding purified water to the precipitate of step (3), and then stirring and dissolving the resulting mixture; and adding a C1-C4 alcohol to the lysate and centrifuging the resulting mixture to obtain a precipitate.

In an embodiment, the ratio of the lysate to the C1-C4 alcohol added to the lysate may be 1:1 to 3 by volume. Specifically, the volume ratio of the lysate to the C1-C4 alcohol added to the lysate may be 1:1, 1:1.1, 1:1.2, 1:1.3, 1:1.4, 1:1.5, 1:1.6, 1:1.7, 1:1.8, 1:1.9, 1:2, 1:2. 1, 1:2.2, 1:2.3, 3:7, 1:2.4, 1:2.5, 1:2.6, 1:2.7, 1:2.8, 1:2.9 or 1:3.

In an embodiment, the C1-C4 alcohol of step (3) and the C1-C4 alcohol of step (4) may be different from each other.

In an embodiment, the C1-C4 alcohol may each be independently methanol, ethanol, propyl alcohol, isopropyl alcohol, or butanol.

In an embodiment, the method may further include (5) freeze-drying the precipitate of step (4).

In an embodiment, in step (5), purified water may be added to the precipitate of step (4) to freeze-dry a solution obtained by stirring and dissolving the resulting mixture.

In still another aspect, the present invention may relate to a cosmetic composition including the PDRN extract as an active ingredient.

The cosmetic composition according to an embodiment of the present invention may contain a cosmetically or dermatologically acceptable medium or base. It is any formulation suitable for topical application, and may be provided in the form of, for example, a solution, a gel, a solid, a kneaded anhydrous product, an emulsion obtained by dispersing an oil phase in the form of an aqueous phase, a suspension, a microemulsion, a microcapsule, microgranules or an ionic (liposome) and a non-ionic vesicular dispersing agent, or in the form of a cream, a skin toner, a lotion, a powder, an ointment, a spray, or a conceal stick. These compositions may be prepared by a typical method in the field. The cosmetic composition may also be used in the form of a foam or an aerosol composition that further contains a compressed propellant.

The formulation of the cosmetic composition is not particularly limited, and may be appropriately selected depending on the purpose. For example, the cosmetic composition may be prepared into a formulation such as a skin lotion, a skin softener, a skin toner, a lotion, a milk lotion, a moisture lotion, a nourishing lotion, a massage cream, a nourishing cream, a moisture cream, a hand cream, a foundation, an essence, a nourishing essence, a pack, a soap, a cleansing foam, a cleansing lotion, a cleansing cream, a cleansing water, a powder, a body lotion, a body cream, a body oil, a body cleanser and a body essence.

When the formulation of the cosmetic composition is a paste, a cream or a gel, an animal oil, a vegetable oil, a wax, paraffin, starch, traganth, a cellulose derivative, a polyethylene glycol, silicone, bentonite, silica, talc, zinc oxide, or the like may be used as a carrier ingredient.

When the formulation of the cosmetic composition is a powder or a spray, lactose, talc, silica, aluminum hydroxide, calcium silicate, or a polyamide powder may be used as the carrier ingredient, and in particular, when the formulation of the present invention is a spray, the formulation may additionally include a propellant such as a chlorofluorohydrocarbon, propane/butane or dimethyl ether.

When the formulation of the cosmetic composition is a solution or an emulsion, a solvent, a solubilizer or an emulsifier is used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the cosmetic composition is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, traganth, or the like may be used as a carrier ingredient.

When the formulation of the cosmetic composition is a surfactant-containing cleanser, an aliphatic alcohol sulfate, an aliphatic alcohol ether sulfate, sulphosuccinic acid monoester, isethionate, an imidazolinium derivative, methyltaurate, sarcosinate, fatty acid amide ether sulfate, alkylamidobetaine, an aliphatic alcohol, fatty acid glyceride, fatty acid diethanolamide, a vegetable oil, a lanolin derivative, an ethoxylated glycerol fatty acid ester, or the like may be used as the carrier ingredient.

The cosmetic composition may further include functional additives and ingredients included in general cosmetic compositions in addition to the green tea peptide. The functional additive may include an ingredient selected from the group consisting of a water-soluble vitamin, an oil-soluble vitamin, a polymeric peptide, a polymeric polysaccharide, a sphingolipid, and a seaweed extract.

The cosmetic composition may also be blended with ingredients included in general cosmetic compositions, if necessary, along with the functional additives. Examples of other blended ingredients which may be contained include oil and fat ingredients, a moisturizer, an emollient, a surfactant, organic and inorganic pigments, an organic powder, an ultraviolet absorbent, a preservative, a bactericide, an antioxidant, a plant extract, a pH adjuster, an alcohol, a colorant, a fragrance, a circulation accelerator, a cooling agent, an antiperspirant, purified water, and the like.

In yet another aspect, the present invention may relate to an injection composition including the PDRN extract as an active ingredient.

In an embodiment, the injection composition may be an injection composition for tissue repair.

In an embodiment, the injection composition may be injected into the dermis layer of the skin.

In still yet another aspect, the present invention may relate to a food composition, a health functional food composition, or a non-therapeutic oral composition including the PDRN extract as an active ingredient.

In an embodiment, a formulation of the food composition or non-therapeutic oral composition is not particularly limited, but the food composition or non-therapeutic oral composition may be formulated into, for example, a tablet, a granule, a pill, a powder, a liquid such as a drink, a caramel, a gel, a bar, a tea bag, or the like. For the composition of each formulation, ingredients typically used in the art may be appropriately selected and compounded by a person with ordinary skill in the art according to the dosage form or purpose of use, in addition to the active ingredient, and when the composition is simultaneously applied with other raw materials, a synergistic effect may occur.

In an embodiment, the food composition or non-therapeutic oral composition may be administered by various methods such as simple ingestion, drinking, injection administration, spray administration, or squeeze administration.

In an embodiment, the food composition or non-therapeutic oral composition may be, for example, various foods such as chewing gum, chocolate, caramel products, candies, frozen treats, and confectionery, beverage products such as soft drinks, mineral water, and alcoholic beverages, and health functional foods including vitamins or minerals.

In an embodiment, the food composition or non-therapeutic oral composition may be taken as is or used together with other foods or food ingredients, and may be appropriately used by a typical method. The food composition or non-therapeutic oral composition may include a sitologically acceptable food supplement additive in addition to the PDRN extract, and the amount of the active ingredient mixed may be appropriately determined depending on the purpose of use.

As used herein, the “food supplement additive” refers to a component that can be added to food as a supplement, and is added to prepare a health functional food of each formulation, and may be appropriately selected and used by those skilled in the art. Examples of the food supplement additive include various nutrients, vitamins, minerals (electrolytes), flavoring agents such as synthetic flavoring agents and natural flavoring agents, colorants and fillers, pectic acid and salts thereof, alginic acid and salts thereof, organic acids, protective colloid thickeners, pH adjusting agents, stabilizers, preservatives, glycerin, alcohols, carbonating agents used in carbonated beverages, and the like, but the types of food supplement additives of the present invention are not limited by the above examples.

As used herein, the “health functional food” refers to a food prepared and processed in the form of a tablet, capsule, powder, granule, liquid, pill, and the like using raw materials or ingredients that have functionality useful to the human body. Here, ‘functionality’ means that useful effects for health applications such as regulating nutrients and physiological actions are obtained for the structure and function of the human body. The health functional food of the present invention can be prepared by a method typically used in the art, and may be prepared by adding raw materials and ingredients typically added in the art during preparation. In addition, the formulation of the health functional food may also be prepared without limitation as long as it is recognized as a health functional food. The food composition may be prepared in various forms of formulations.

The present invention may provide the following embodiments as an example.

A first embodiment may provide a high-purity polydeoxyribonucleotide (PDRN) extract derived from microalgae, wherein a DNA content in the PDRN extract relative to the total dry weight of the PDRN extract is 49 wt % or more.

A second embodiment may provide the PDRN extract of the first embodiment, wherein the DNA content in the PDRN extract relative to the total dry weight of the PDRN extract is 70 wt % or more.

A third embodiment may provide the PDRN extract of one or more of the first and second embodiments, wherein at least 90% of the DNA in the PDRN extract is low molecular weight DNA having a molecular weight of 20 kDa or less.

A fourth embodiment may provide the PDRN extract of one or more of the first to third embodiments, wherein the PDRN extract is in the form of a white powder.

A fifth embodiment may provide the PDRN extract of one or more of the first to fourth embodiments, wherein the microalgae is one or more selected from a group consisting of golden chlorella (Chlorella protothecoides), green chlorella (Chlorella vulgaris), white chlorella (Chlorella protothecoides), Spirulina, Dunaliella, Haematococcus, Euglena, Nannochloropsis, Nostoc, and Schizochytrium.

A sixth embodiment may provide a method for preparing a high-purity PDRN extract derived from microalgae, the method including: (1) adding an anionic surfactant and a metal salt to a microalgae solution and stirring the resulting mixture to obtain a cytolysate;

(2) adding a metal salt to a filtrate obtained by centrifuging and filtering the cytolysate of step (1) to stir the resulting mixture;

(3) a first purification step of adding a C1-C4 alcohol to a filtrate obtained by centrifuging and filtering the stirred solution of step (2) and centrifuging the resulting mixture to obtain a precipitate; and

(4) a second purification step of adding a C1-C4 alcohol to the precipitate of step (3) and centrifuging the resulting mixture to obtain a precipitate.

A seventh embodiment may provide the preparation method of the sixth embodiment, wherein the microalgae solution of step (1) is a mixture of a microalgae powder and purified water, and the weight of the microalgae solution is 20-fold or more than the weight of the microalgae powder.

An eighth embodiment may provide the preparation method of one or more of the sixth and seventh embodiments, wherein the microalgae of step (1) is one or more selected from a group consisting of golden chlorella (Chlorella protothecoides), green chlorella (Chlorella vulgaris), white chlorella (Chlorella protothecoides), Spirulina, Dunaliella, Haematococcus, Euglena, Nannochloropsis, Nostoc, and Schizochytrium.

A ninth embodiment may provide the preparation method of one or more of the sixth to eighth embodiments, wherein the anionic surfactant of step (1) is sodium dodecyl sulfate (SDS).

A tenth embodiment may provide the preparation method of one or more of the sixth to ninth embodiments, further including repeatedly applying an ultra-high pressure of 500 to 2,000 bar to the mixture of step (1) three or more times to obtain cell debris, prior to step (2).

An eleventh embodiment may provide the preparation method of one or more of the sixth to tenth embodiments, wherein the metal salt is NaCl.

A twelfth embodiment may provide the preparation method of one or more of the sixth to eleventh embodiments, wherein the concentration of the metal salt added in step (2) is 2-fold or more than the concentration of the metal salt added in step (1).

A thirteenth embodiment may provide the preparation method of one or more of the sixth to twelfth embodiments, wherein the metal salt and a cationic surfactant are added together in step (2).

A fourteenth embodiment may provide the preparation method of one or more of the sixth to thirteenth embodiments, wherein the cationic surfactant is cetyl trimethyl ammonium bromide (CTAB).

A fifteenth embodiment may provide the preparation method of one or more of the sixth to fourteenth embodiments, wherein the C1-C4 alcohol of step (3) and the C1-C4 alcohol of step (4) are different from each other.

A sixteenth embodiment may provide a cosmetic composition including the PDRN extract of any one of the first to fifth embodiments or a PDRN extract prepared by any one method of the sixth to fifteenth embodiments as an active ingredient.

A seventeenth embodiment may provide a food composition including the PDRN extract of any one of the first to fifth embodiments or a PDRN extract prepared by any one method of the sixth to fifteenth embodiments as an active ingredient.

Hereinafter, the contents of the present invention will be more specifically described through Examples and Test Examples. However, these Examples and Test Examples are presented merely for the purpose of understanding the contents of the present invention, and the scope of the present invention is not limited to these Examples and Test Examples, modifications, substitutions, insertions, and the like that are typically known in the art may be made, and such modifications are also included in the scope of the present invention.

### [Preparation Example 1] Preparation of Golden Chlorella-Derived PDRN Extract

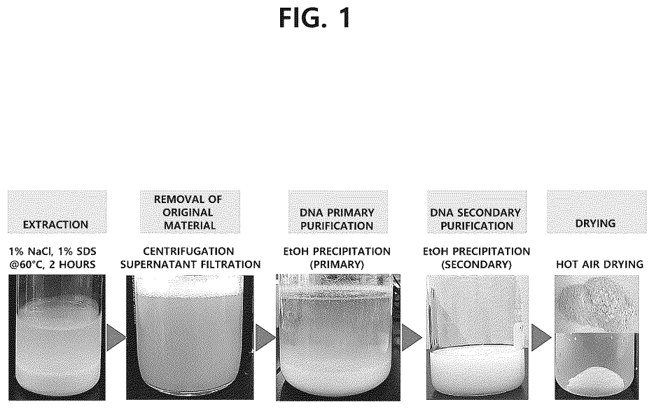

The process of preparing a PDRN extract from golden chlorella is schematically shown in FIG. 1. Specifically, 900 g of purified water was added to 100 g of dried golden chlorella powder (DAESANG), and then the resulting mixture was placed in a stirrer and homogenized at 25° C. to 30° C. for 30 minutes. After sodium dodecyl sulfate (SDS) and sodium chloride (NaCl) were added to the homogenate to a concentration of 1% (w/w), respectively, the cell lysis process was carried out with stirring at 60° C. for 2 hours. After the cytolysate was centrifuged at 7000 rpm for 30 minutes using a centrifuge, the supernatant was obtained. The obtained supernatant was filtered in steps from 1 μm to 0.45 μm to 0.2 μm. Ethanol was added to this filtrate, such that the ratio of filtrate to ethanol was 30:70 by volume, the resulting mixture was allowed to precipitate under the conditions of 0° C. to 4° C. overnight, and then a precipitate was obtained using a centrifuge (5000 rpm, 10 minutes). After 100 g of purified water was added to this precipitate, the precipitate was redissolved with stirring at room temperature for 30 minutes. Ethanol was added to this lysate, such that the ratio of lysate to ethanol was 30:70 by volume, the resulting mixture was allowed to precipitate under the conditions of 0° C. to 4° C. overnight, and then a precipitate was obtained using a centrifuge (5000 rpm, 10 minutes). Thereafter, the precipitate was dried with hot air at room temperature for 3 hours to obtain a golden chlorella-derived PDRN extract.

After the obtained PDRN extract was dissolved in purified water to a concentration of 10 mg/ml, the concentration and purity of DNA in the PDRN extract were measured using a Nanodrop-1000 instrument (Thermo-Fisher, Wilmington, DE, USA). DNA purity was evaluated by the ratio of absorbance at 260 nm to 280 nm and the ratio of absorbance at 260 nm to 230 nm, and generally, it is determined that when a 260/280 ratio is 1.8 or higher, the DNA has good purity with minimal contamination by proteins and the like, and that when a 260/230 ratio is 1.8 or higher, the DNA has good purity with minimal contamination by polysaccharides, polyphenols, and the like. The results are shown in the following Table 1.

TABLE 1

| | | | | | Amount of | Amount of |
|---|---|---|---|---|---|---|
| | | | | | PDRN | DNA |
| Concentration | | | | | extract | obtained |
| of PDRN | | | | | obtained | compared |
| extract | DNA | DNA | | | compared to | to original |
| dissolved | concentration | content | ratio | Ratio | original | material |
| (mg/ml) | (μg/ml) | (%) | (260/280) | (260/230) | material (%) | (%) |
| 10 | 1069.4 | 10.69 | 1.99 | 1.21 | 15.19 | 1.62 |

From the results in Table 1, it was confirmed that proteins were well removed when the 260/280 ratio was 1.99, but polysaccharides and the like were not removed and the purity was low when the 260/230 ratio was measured as low at 1.21, and that the purity of DNA in the PDRN extract was also low when the DNA content in the PDRN extract was about 10.7%.

### [Test Example 1] Evaluation of Cell Lysis Effect According to Anionic Surfactant Concentration

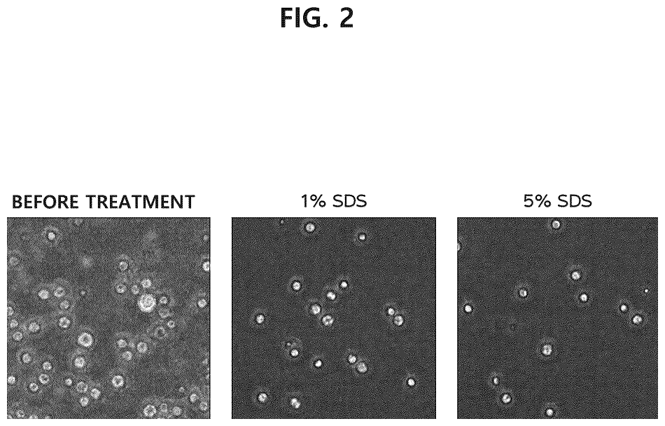

900 g of purified water was added to 100 g of dried golden chlorella powder (DAESANG), and then the resulting mixture was placed in a stirrer and homogenized at 25° C. to 30° C. for 30 minutes. After sodium dodecyl sulfate (SDS) and sodium chloride (NaCl) were added to the homogenate to a concentration of 1% (w/w), respectively, and 5% (w/w) and 1% (w/w), respectively, the cell lysis process was carried out with stirring at 60° C. for 2 hours. Thereafter, the cell morphology was observed under an optical microscope (SZX16, OLYMPUS, JAPAN), and the results are shown in FIG. 2.

From the results in FIG. 2, it could be confirmed that the cell lysis effect of chlorella is enhanced even though the amount of anionic surfactant (SDS) used is not increased.

### [Preparation Example 2] Preparation of Golden Chlorella-Derived PDRN Extract Using Ultra-High Pressure Disperser

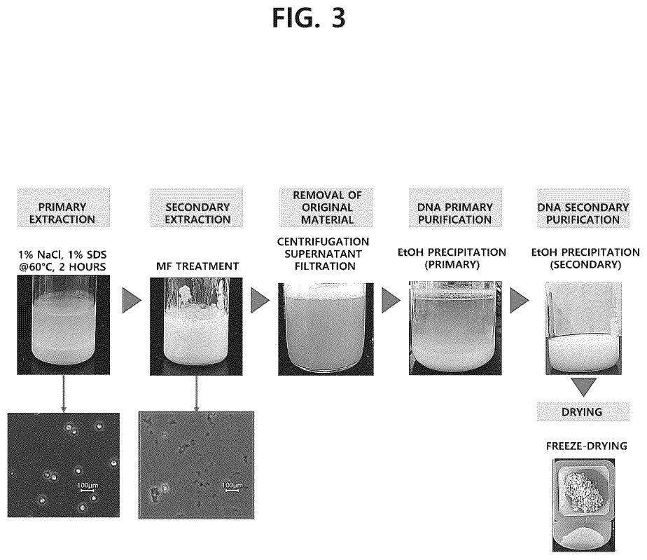

The process of preparing a PDRN extract from golden chlorella using an ultra-high pressure disperser is schematically shown in FIG. 3. Specifically, 900 g of purified water was added to 100 g of dried golden chlorella powder (DAESANG), and then the resulting mixture was placed in a stirrer and homogenized at 25° C. to 30° C. for 30 minutes. After SDS and NaCl were added to the homogenate to a concentration of 1%, respectively, the cell lysis process was carried out with stirring at 60° C. for 2 hours. Cells were disrupted while treating the cytolysate three times with an ultra-high pressure disperser (M110EH Microfluidizer, Microfluidics International Corporation, USA) at a pressure of 1500 bar. It was confirmed that the cell debris had a creamy texture, as shown in FIG. 3, and as a result of observing the morphology of the chlorella cells under an optical microscope after the first and second extractions, respectively, it could be confirmed that the cells had been completely lysed, as shown in FIG. 3. Thereafter, the cell debris was centrifuged at 7000 rpm for 20 minutes using a centrifuge, and the supernatant was obtained. The obtained supernatant was filtered in steps from 1 μm to 0.45 μm to 0.2 μm. Ethanol was added to this filtrate, such that the ratio of filtrate to ethanol was 30:70 by volume, the resulting mixture was allowed to precipitate under the conditions of 0° C. to 4° C. for 1 hour, and then a precipitate was obtained using a centrifuge (5000 rpm, 10 minutes). After 100 g of purified water was added to this precipitate, the precipitate was redissolved with stirring at room temperature for 30 minutes. Ethanol was added to this lysate, such that the ratio of lysate to ethanol was 30:70 by volume, the resulting mixture was allowed to precipitate under the conditions of 0° C. to 4° C. for 1 hour, and then a precipitate was obtained using a centrifuge (5000 rpm, 10 minutes). Thereafter, 100 g of purified water was added to this precipitate, and the precipitate was redissolved with stirring at room temperature for 30 minutes. The solution was dried in a freeze dryer for 2 days to obtain a golden chlorella-derived PDRN extract using an ultra-high pressure disperser.

After the obtained PDRN extract was dissolved in purified water to a concentration of 10 mg/ml, the concentration and purity of DNA in the PDRN extract were measured using a Nanodrop-1000 instrument (Thermo-Fisher, Wilmington, DE, USA). The results are shown in the following Table 2.

TABLE 2

| | | | | | Amount of | Amount of |
|---|---|---|---|---|---|---|
| | | | | | PDRN | DNA |
| Concentration | | | | | extract | obtained |
| of PDRN | | | | | obtained | compared |
| extract | DNA | DNA | | | compared to | to original |
| dissolved | concentration | content | ratio | Ratio | original | material |
| (mg/ml) | (μg/ml) | (%) | (260/280) | (260/230) | material (%) | (%) |
| 10 | 1672 | 16.72 | 1.87 | 1.14 | 20.69 | 3.46 |

From the results in Table 2, it was confirmed that even when the ultra-high pressure disperser was used, proteins were well removed when the 260/280 ratio was 1.87, as in Preparation Example 1, but polysaccharides and the like were not removed and the purity was low when the 260/230 ratio was measured as low at 1.14. The DNA content in the PDRN extract was about 16.7%, and it was confirmed that the purity of DNA in the PDRN extract was increased compared to Preparation Example 1, but was still at a low level.

### [Preparation Example 3] Preparation of Golden Chlorella-Derived PDRN Extract Using High Concentration of Metal Salt

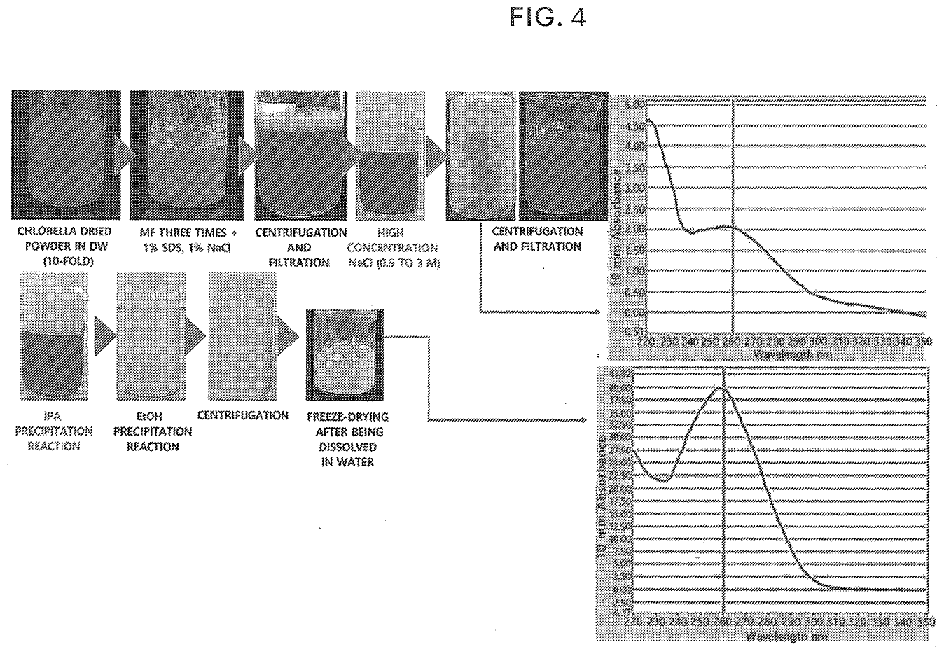

The process of preparing a PDRN extract from golden chlorella additionally using a high concentration of a metal salt is schematically shown in FIG. 4. Specifically, 900 g of purified water was added to 100 g of dried golden chlorella powder (DAESANG), and then the resulting mixture was placed in a stirrer and homogenized at 25° C. to 30° C. for 30 minutes. After SDS and NaCl were added to the homogenate to a concentration of 1%, respectively, the cell lysis process was carried out with stirring at 60° C. for 2 hours. Cells were disrupted while repeatedly treating the cytolysate three times with an ultra-high pressure disperser at a pressure of 1500 bar. Thereafter, the cell debris was centrifuged at 7000 rpm for 20 minutes using a centrifuge, and the supernatant was obtained. The obtained supernatant was filtered in steps from 1 μm to 0.45 μm to 0.2 μm. NaCl was added to the filtrate such that the concentration became 0.5 M, 1 M, 1.5 M, 2 M, and 2.5 M, respectively, and the resulting mixture was stirred at room temperature for 1 hour. Thereafter, the mixture was centrifuged at 7000 rpm for 20 minutes using a centrifuge, and the supernatant was obtained. The obtained supernatant was filtered in steps from 1 μm to 0.45 μm to 0.2 μm. Isopropyl alcohol (IPA) was added to this filtrate, such that the ratio of filtrate to IPA was 50:50 by volume, the resulting mixture was allowed to precipitate under the conditions of 0° C. to 4° C. for 1 hour, and then a precipitate was obtained using a centrifuge (5000 rpm, 10 minutes). After 100 g of purified water was added to this precipitate, the precipitate was redissolved with stirring at room temperature for 30 minutes. Ethanol was added to this lysate, such that the ratio of lysate to ethanol was 30:70 by volume, the resulting mixture was allowed to precipitate under the conditions of 0° C. to 4° C. for 1 hour, and then a precipitate was obtained using a centrifuge (5000 rpm, 10 minutes). Thereafter, 100 g of purified water was added to this precipitate, and the precipitate was redissolved with stirring at room temperature for 30 minutes. The solution was dried in a freeze dryer for 2 days to obtain a golden chlorella-derived PDRN extract.

After the obtained PDRN extract was dissolved in purified water to a concentration of 1 mg/ml or 10 mg/ml, the concentration and purity of DNA in the PDRN extract were measured using a Nanodrop-1000 instrument (Thermo-Fisher, Wilmington, DE, USA). The results are shown in the following Table 3.

TABLE 3

| | | | | | | Amount of | Amount of |
|---|---|---|---|---|---|---|---|
| | | | | | | PDRN | DNA |
| | Concentration | | | | | extract | obtained |
| | of PDRN | | | | | obtained | compared |
| | extract | DNA | DNA | | | compared to | to original |
| NaCl | dissolved | concentration | content | ratio | Ratio | original | material |
| Concentration | (mg/ml) | (μg/ml) | (%) | (260/280) | (260/230) | material (%) | (%) |
| 0.5M | 10 | 2341 | 23.41 | 1.94 | 1.27 | 19.60 | 4.59 |
| 1M | 10 | 2147 | 21.47 | 1.93 | 1.48 | 19.84 | 4.26 |
| 1.5M | 10 | 2860 | 28.60 | 2.02 | 1.89 | 6.28 | 1.80 |
| 2M | 1 | 495 | 49.5 | 1.99 | 1.83 | 2.84 | 1.40 |
| 2.5M | 1 | 490 | 49.0 | 2.01 | 1.89 | 2.40 | 1.18 |

From the results in Table 3, it could be confirmed that upon treatment with NaCl at a concentration of 1.5 M, 2 M, and 2.5 M, contamination with polysaccharides and the like was removed when the 260/230 ratio value was 1.8 or higher. In addition, when the NaCl treatment concentration was high at 2 M and 2.5 M, the DNA content in the chlorella PDRN extract was confirmed to be about 50%, confirming that the purity of DNA in the PDRN extract was significantly increased. Furthermore, it could be confirmed that as the purity of DNA in the PDRN extract was increased, a PDRN extract in the form of a white powder could be obtained, unlike Preparation Examples 1 and 2, which were obtained in the form of a yellow powder, and unpleasant odors such as a fishy smell were also significantly reduced.

### [Preparation Example 4] Preparation of Golden Chlorella-Derived PDRN Extract Using Cationic Surfactant

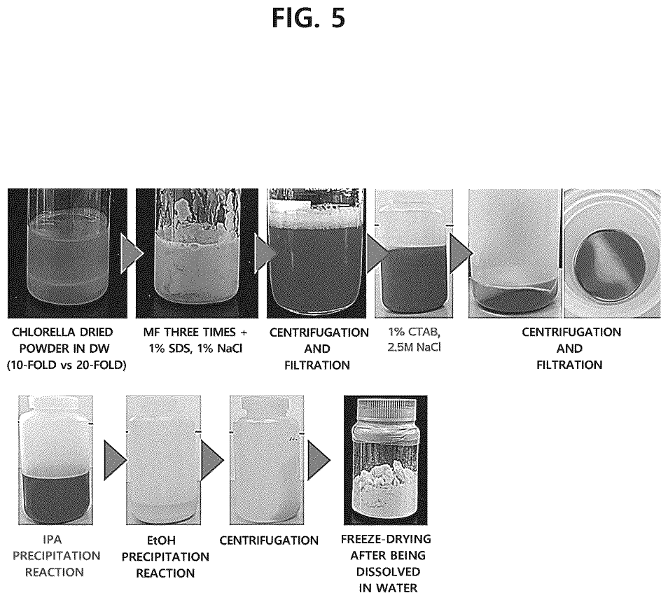

The process of preparing a PDRN extract from golden chlorella additionally using a cationic surfactant is schematically shown in FIG. 5. Specifically, 900 g of purified water was added to 100 g of dried golden chlorella powder (DAESANG) (Preparation Example 4-1) or 950 g of purified water was added to 50 g of dried golden chlorella powder (DAESANG) (Preparation Example 4-2), and then the resulting mixture was placed in a stirrer and homogenized at 25° C. to 30° C. for 30 minutes. After SDS and NaCl were added to the homogenate to a concentration of 1%, respectively, the cell lysis process was carried out with stirring at 60° C. for 2 hours. Cells were disrupted while repeatedly treating the cytolysate three times with an ultra-high pressure disperser at a pressure of 1500 bar. Thereafter, the cell debris was centrifuged at 7000 rpm for 20 minutes using a centrifuge, and the supernatant was obtained. The obtained supernatant was filtered in steps from 1 μm to 0.45 μm to 0.2 μm. Cetyl trimethyl ammonium bromide (CTAB) and NaCl were added to the filtrate such that the concentration became 1% (w/w) and 2.5 M, respectively, and the resulting mixture was stirred at room temperature for 1 hour. Thereafter, the mixture was centrifuged at 7000 rpm for 20 minutes using a centrifuge, and the supernatant was obtained. The obtained supernatant was filtered in steps from 1 μm to 0.45 μm to 0.2 μm. Isopropyl alcohol (IPA) was added to this filtrate, such that the ratio of filtrate to IPA was 50:50 by volume, the resulting mixture was allowed to precipitate under the conditions of 0° C. to 4° C. for 1 hour, and then a precipitate was obtained using a centrifuge (5000 rpm, 10 minutes). After 100 g of purified water was added to this precipitate, the precipitate was redissolved with stirring at room temperature for 30 minutes. Ethanol was added to this lysate, such that the ratio of lysate to ethanol was 30:70 by volume, the resulting mixture was allowed to precipitate under the conditions of 0° C. to 4° C. for 1 hour, and then a precipitate was obtained using a centrifuge (5000 rpm, 10 minutes). Thereafter, 100 g of purified water was added to this precipitate, and the precipitate was redissolved with stirring at room temperature for 30 minutes. The solution was dried in a freeze dryer for 2 days to obtain a golden chlorella-derived PDRN extract.

After the obtained PDRN extract was dissolved in purified water to a concentration of 1 mg/ml, the concentration and purity of DNA in the PDRN extract were measured using a Nanodrop-1000 instrument (Thermo-Fisher, Wilmington, DE, USA). The results are shown in the following Table 4.

TABLE 4

| | | | | | | Amount of | Amount of |
|---|---|---|---|---|---|---|---|
| | | | | | | PDRN | DNA |
| | Concentration | | | | | extract | obtained |
| | of PDRN | | | | | obtained | compared |
| | extract | DNA | DNA | | | compared to | to original |
| | dissolved | concentration | content | ratio | Ratio | original | material |
| | (mg/ml) | (μg/ml) | (%) | (260/280) | (260/230) | material (%) | (%) |
| Preparation | 1 | 579 | 57.93 | 2.01 | 1.99 | 3.13 | 1.82 |
| Example | | | | | | | |
| 4-1 | | | | | | | |
| Preparation | 1 | 814 | 81.43 | 2.13 | 2.23 | 3.20 | 2.61 |
| Example | | | | | | | |
| 4-2 | | | | | | | |

From the results in Table 4, it could be confirmed that the content of DNA in the PDRN extract was higher in Preparation Example 4, in which a cationic surfactant was additionally used, than that in Preparation Example 3. In particular, in the case of Preparation Example 4-2, in which the weight of the cell lysis solution (dried chlorella powder+purified water) was 20-fold that of the dried chlorella powder, it could be confirmed that DNA with a remarkably high purity could be extracted. As a result, when a cell lysis solution 20-fold higher than the original material (dried chlorella powder) was treated with a cationic surfactant and a high concentration of a metal salt, a chlorella PDRN with a high DNA content of about 80% or more could be obtained.

### [Test Example 2] Comparison of PDRN Extraction Yield and DNA Purity Using Ultra-High Pressure Disperser

In order to confirm whether the extraction yield and DNA purity of the chlorella PDRN change according to the presence and absence of the process of disrupting cells using an ultra-high pressure disperser, a chlorella PDRN was obtained in the same manner as in Preparation Example 4-2, except that the cell disruption process by treatment using an ultra-high pressure disperser was not performed (Preparation Example 4-3), and the DNA purity and PDRN extraction yield were confirmed, and the results are shown in the following Table 5.

TABLE 5

| | | | | | | Amount of | Amount of |
|---|---|---|---|---|---|---|---|
| | | | | | | PDRN | DNA |
| | Concentration | | | | | extract | obtained |
| | of PDRN | | | | | obtained | compared |
| | extract | DNA | DNA | | | compared to | to original |
| | dissolved | concentration | content | ratio | Ratio | original | material |
| | (mg/ml) | (μg/ml) | (%) | (260/280) | (260/230) | material (%) | (%) |
| Preparation | 1 | 814 | 81.43 | 2.13 | 2.23 | 3.20 | 2.61 |
| Example 4-2 | | | | | | | |
| Preparation | 1 | 791 | 79.10 | 2.1 | 2.17 | 1.02 | 0.90 |
| Example 4-3 | | | | | | | |

From the results in Table 5, it was confirmed that in the case of Preparation Example 4-3, which was not subjected to the ultra-high pressure dispersion process, the purity of DNA in the PDRN extract was about 79%, which was similar to that of Preparation Example 4-2.However, it was found that the yield of the PDRN extract was very low at about 1% compared to the original material, and it could be confirmed that when an ultra-high pressure disperser was not used, it was disadvantageous in terms of raw material yield.

### [Preparation Example 5] Extraction of PDRN derived from green chlorella and white chlorella

PDRN extracts were obtained in the same manner as in Preparation Example 4-2, except that dried green chlorella powder (Preparation Example 5-1) and dried white chlorella powder (Preparation Example 5-2) were used instead of dried golden chlorella powder.

After the obtained PDRN extract was dissolved in purified water to a concentration of 1 mg/ml, the concentration and purity of DNA in the PDRN extract were measured using a Nanodrop-1000 instrument (Thermo-Fisher, Wilmington, DE, USA). The results are shown in the following Table 6.

TABLE 6

| | | | | | | Amount of | Amount of |
|---|---|---|---|---|---|---|---|
| | | | | | | PDRN | DNA |
| | Concentration | | | | | extract | obtained |
| | of PDRN | | | | | obtained | compared |
| | extract | DNA | DNA | | | compared to | to original |
| | dissolved | concentration | content | ratio | Ratio | original | material |
| | (mg/ml) | (μg/ml) | (%) | (260/280) | (260/230) | material (%) | (%) |
| Preparation | 1 | 814 | 81.43 | 2.13 | 2.23 | 3.20 | 2.61 |
| Example | | | | | | | |
| 4-2 | | | | | | | |
| Preparation | 1 | 820.76 | 82.08 | 2.13 | 2.32 | 3.05 | 2.50 |
| Example | | | | | | | |
| 5-1 | | | | | | | |
| Preparation | 1 | 478.22 | 47.82 | 2.09 | 2.19 | 3.04 | 1.45 |
| Example | | | | | | | |
| 5-2 | | | | | | | |

From the results in Table 6, it was confirmed that the 260/280 ratio and 260/230 ratio values of all the PDRN extracts extracted from the three types of chlorella (golden, green, and white) were measured to be 2 or higher, and therefore, had extremely high purity with almost all contaminants such as proteins and polysaccharides removed, and it could be confirmed that the DNA content in the PDRN extracts was extremely high, at about 81% and 82% for the PDRNs derived from golden chlorella and green chlorella, respectively.

### [Test Example 3] Comparison of Size and Purity of PDRN Derived from Three Types of Chlorella

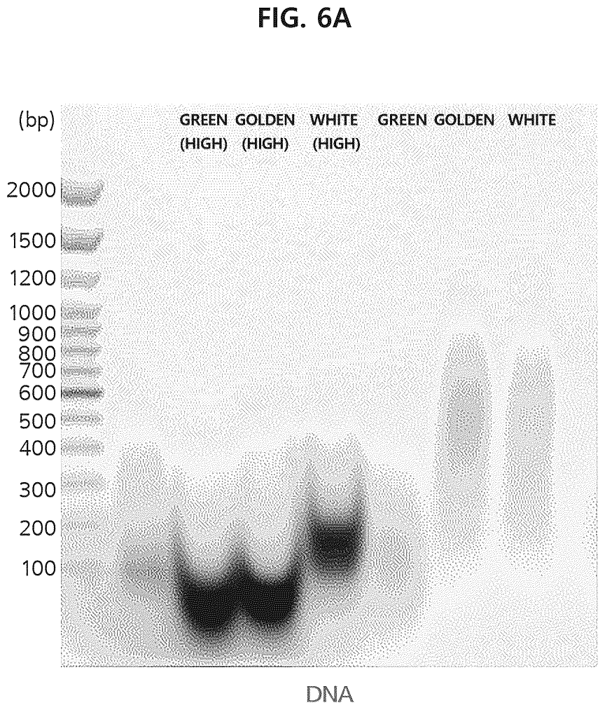

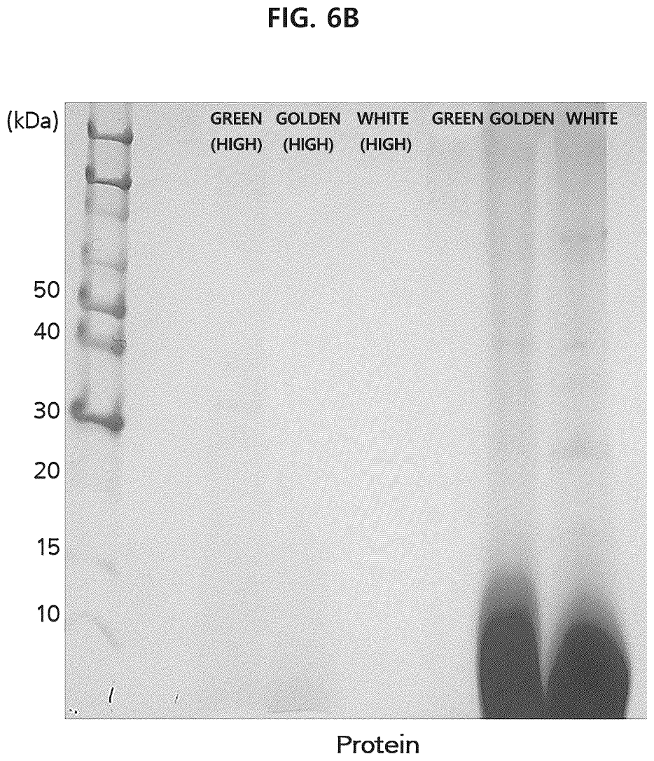

Electrophoresis was performed to measure the size of each of the PDRNs of Preparation Example 4-2 (golden chlorella-derived PDRN), Preparation Example 5-1 (green chlorella-derived PDRN), and Preparation Example 5-2 (white chlorella-derived PDRN). Specifically, each PDRN extract was dissolved in a DNA gel loading buffer (ThermoSCIENTIFIC, USA) at a concentration of 10 mg/ml, and then electrophoresis was performed using a 1% agarose gel, which is a level capable of separating DNA fragments ranging from 200 bp to 50 kb. For comparison, a chlorella PDRN raw material extracted with a DNA content ranging from 10% to 20% was dissolved at the same concentration (10 mg/ml), and then electrophoresis was performed. The results are shown in FIG. 6A. As shown in FIG. 6A, it could be seen that the high-purity green and golden chlorella PDRNs were a mixture of DNA fragments of 100 bp or less, while the white chlorella PDRN was a mixture of DNA fragments corresponding to a level of 100 bp to 200 bp. In contrast, it could be seen that the three chlorella PDRNs with a purity of 10% to 20% were confirmed in a wide range of 100 bp to 700 bp, and showed a very low band intensity compared to the high-purity PDRNs. It is presumed that this DNA forms a complex with these components due to contamination with proteins, polysaccharides, and the like during the PDRN extraction process, and it is determined that this shows the larger DNA size, lower band intensity, and band trailing phenomenon in electrophoresis.

Additionally, to compare the degree of protein contamination of the three chlorella PDRNs extracted with high and low purity, electrophoresis was performed in the same manner as above, except that a 12% acrylamide gel was used, and the results are shown in FIG. 6B. As shown in FIG. 6B, it was confirmed that there was almost no protein contamination in the case of the three high-purity chlorella PDRNs. In contrast, the golden and white chlorella PDRNs with a purity of 10% to 20% had a large amount of protein contamination, most of which were confirmed to 10 kDa or less in size.

In conclusion, it could be confirmed that the PDRNs of Preparation Example 4-2 (golden chlorella-derived PDRN), Preparation Example 5-1 (green chlorella-derived PDRN), and Preparation Example 5-2 (white chlorella-derived PDRN) were mixtures of low molecular weight DNA fragments in which protein contamination and the like were minimized.

### [Test Example 4] Comparison of Size and Purity of PDRN Derived from High-Purity Golden Chlorella

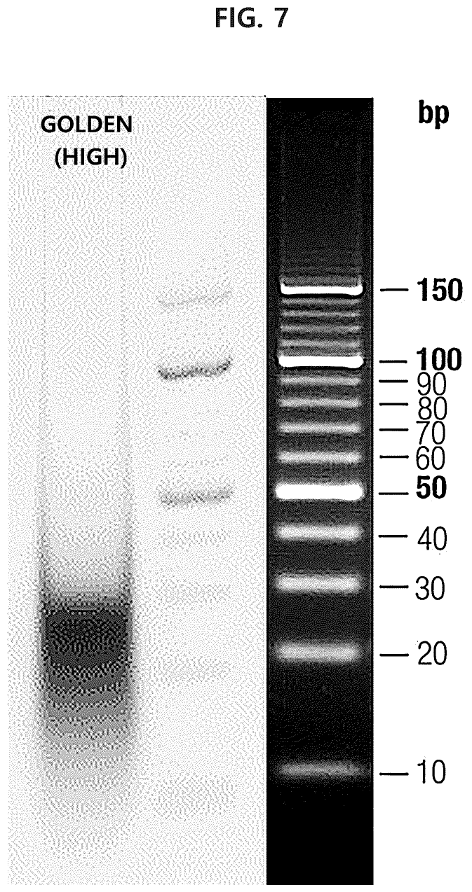

To more accurately confirm the DNA size of the PDRN derived from golden chlorella in Preparation Example 4-2, the PDRN extract derived from golden chlorella in Preparation Example 4-2 was dissolved in a DNA gel loading buffer at a concentration of 10 mg/ml, and then electrophoresis was performed using a 5% agarose gel. The results are shown in FIG. 7.

As shown in FIG. 7, it was confirmed that the size of DNA in the golden chlorella-derived PDRN extract of Preparation Example 4-2 was be 20 bp to 30 bp, and it could be confirmed that the molecular weight of DNA in the high-purity golden chlorella-derived PDRN according to an embodiment of the present invention was at a level of 12 to 18 kDa, which is a low molecular weight remarkably lower than the molecular weight range of DNA in an existing salmon PDRN (50 to 1500 kDa) because the average molecular weight of one base pair was 650 Daltons in the molecular weight calculation of DNA.

We claim:

1. A high-purity polydeoxyribonucleotide (PDRN) extract derived from microalgae, wherein a DNA content in the PDRN extract relative to a total dry weight of the PDRN extract is 49 wt % or more.

2. The PDRN extract of claim 1, wherein the DNA content in the PDRN extract relative to the total dry weight of the PDRN extract is 70 wt % or more.

3. The PDRN extract of claim 1, wherein at least 90% of the DNA in the PDRN extract is low molecular weight DNA having a molecular weight of 20 kDa or less.

4. The PDRN extract of claim 1, wherein the PDRN extract is in a form of a white powder.

5. The PDRN extract of claim 1, wherein the microalgae is one or more selected from a group consisting of golden chlorella (Chlorella protothecoides), green chlorella (Chlorella vulgaris), white chlorella (Chlorella protothecoides), Spirulina, Dunaliella, Haematococcus, Euglena, Nannochloropsis, Nostoc, and Schizochytrium.

6. A method for preparing a high-purity PDRN extract derived from microalgae, the method comprising: (1) adding an anionic surfactant and a metal salt to a microalgae solution and stirring the resulting mixture to obtain a cytolysate;
(2) adding a metal salt to a filtrate obtained by centrifuging and filtering the cytolysate of step (1) to stir the resulting mixture;
(3) a first purification step of adding a C1-C4 alcohol to a filtrate obtained by centrifuging and filtering the stirred solution of step (2) and centrifuging the resulting mixture to obtain a precipitate; and
(4) a second purification step of adding a C1-C4 alcohol to the precipitate of step (3) and centrifuging the resulting mixture to obtain a precipitate.

7. The preparation method of claim 6, wherein the microalgae solution of step (1) is a mixture of a microalgae powder and purified water, and a weight of the microalgae solution is 20-fold or more than a weight of the microalgae powder.

8. The preparation method of claim 6, wherein the microalgae of step (1) is one or more selected from a group consisting of golden chlorella (Chlorella protothecoides), green chlorella (Chlorella vulgaris), white chlorella (Chlorella protothecoides), Spirulina, Dunaliella, Haematococcus, Euglena, Nannochloropsis, Nostoc, and Schizochytrium.

9. The preparation method of claim 6, wherein the anionic surfactant of step (1) is sodium dodecyl sulfate (SDS).

10. The preparation method of claim 6, further comprising repeatedly applying an ultra-high pressure of 500 to 2,000 bar to the mixture of step (1) three or more times to obtain cell debris, prior to step (2).

11. The preparation method of claim 6, wherein the metal salt is NaCl.

12. The preparation method of claim 6, wherein a concentration of the metal salt added in step (2) is 2-fold or more than a concentration of the metal salt added in step (1).

13. The preparation method of claim 6, wherein the metal salt and a cationic surfactant are added together in step (2).

14. The preparation method of claim 13, wherein the cationic surfactant is cetyl trimethyl ammonium bromide (CTAB).

15. The preparation method of claim 6, wherein the C1-C4 alcohol of step (3) and the C1-C4 alcohol of step (4) are different from each other.

16. A cosmetic composition comprising the PDRN extract of claim 1 as an active ingredient.

17. A cosmetic composition comprising a PDRN extract prepared by the method of claim 6 as an active ingredient.

18. A food composition comprising the PDRN extract of claim 1 as an active ingredient.

19. A food composition comprising a PDRN extract prepared by the method of claim 6 as an active ingredient.
